(19) United States
(12) Patent Application Publication
(10) Pub. No.: US 2014/0249647 A1
(43) Pub. Date: Sep. 4, 2014

(54) SYSTEM FOR RECORDING AND PROCESSING NEURAL ACTIVITY

(71) Applicants: Clemens Florian Eder, London (GB); Mads Peter Andersen, Hjorring (DK)

(72) Inventors: Clemens Florian Eder, London (GB); Mads Peter Andersen, Hjorring (DK)

(73) Assignee: NEURODAN A/S, Aalborg SV (DK)

(21) Appl. No.: 14/342,003

(22) PCT Filed: Aug. 27, 2012
(86) PCT No.: PCT/DK2012/050310

§ 371 Date: Apr. 25, 2014

(30) Foreign Application Priority Data
Aug. 29, 2011 (DK) PA 2011 00645

(51) Int. Cl. A61B 5/04; A61N 1/36; A61F 2/72; A61B 5/00

(52) U.S. Cl. CPC A61B 5/04001; A61B 5/4836; A61N 1/36003; A61F 2/72; A61N 1/36007
USPC 623/25; 600/377; 607/49; 607/41

(57) ABSTRACT

A system for recording electroneurographic activity comprising at least three electrodes capable of sensing a nerve signal from a peripheral nerve and having means for receiving and processing the sensed nerve signal to identify a signal indicative of a specific action being a movement of a body part performed by the patient and for producing a control signal in response thereto featuring means for amplifying or attenuating signals in a specific direction of propagation without adversely affecting the electroneurographic activity being measured.

### TECHNICAL FIELD

The present invention is generally concerned with the art of sensing neural signals from nerves. In particular it relates to amplification and processing of nerve activity in order to determine the best timing for initiating electrical stimulation of nerves, or the control of a prosthesis.

### BACKGROUND OF THE INVENTION

Electrical stimulation of nerve trunks and their branches is known to be effective in the treatment of a variety of neurological disorders in humans, spanning from treatment of incontinence to gait disorders. Sensing and recording nerve signals is a discipline that aims for obtaining valuable input for actively controlling the timing of the electrical stimulation of nerves. The recorded nerve signals can also be used for controlling equipment placed outside the body as e.g. prostheses that serve as functional replacement of body parts.

When it comes to the art of electrical stimulation of nerves for the treatment of gait disorders, especially correcting drop-foot, electrodes are placed in the proximity of the peroneal nerve or its branches. An implantable pulse generator connected to the electrode arrangement generates a pattern of pulses to stimulate the nerve which will cause the foot dorsiflexor muscles to contract. Thus the foot will be lifted and it will be possible for the patient to swing the leg more naturally while walking. An example of a system for correction of drop-foot is known from EP 1 257 318 B1 to Neurodan A/S. The document covers the medical aspects and discloses examples of various preferred embodiments. For the triggering of the electrical stimulation of the nerve, according to the wanted reaction of the foot, the use of a heel switch is disclosed. The heel switch can be either connected to the pulse generator with electrical wires or it can include a wireless transmitter module for triggering the pulse generator. For the interface between the pulse generator and the electrodes the system comprises an inductive link, an antenna to be mounted on the skin of the patient and a counterpart in form of an implantable antenna adapted to be implanted in the thigh of the patient. In a further embodiment it is shown that neural information recorded on e.g. the Sural nerve can be used for determining certain gait events such as heel strike and heel lift. For detecting the neural information a nerve recording electrode arrangement is used, where the electrodes -in the preferred embodiment—are being placed inside the wall of an insulating and elastic silicone rubber tube, representing a cuff being wrapped around the nerve. The cuff is used in one embodiment for a multipolar nerve stimulation and recording electrode arrangement, where the electrodes are switched between a mode of recording nerve signals and a mode where electrical nerve stimulation is carried out. As can be seen in FIG. 1, natural sensors can be used as trigger input for a drop foot stimulator. Gait related information can be either sensed from a dedicated sensing electrode arrangement on a purely sensory nerve, or through the same set of electrodes that the mixed common peroneal nerve (sensory and motor branches) is being stimulated with.

When it comes to recording information from natural sensors in living beings, information is encoded as action potentials. These are propagating along nerve fibers, either from their natural sensors, or to their muscles. An action potential is a transient change in the voltage between the intracellular (within the nerve fiber) and extracellular space (outside the nerve fiber) on either side of the membrane, as result of a mechanical, electrical or chemical stimulus that changes the electrochemical balance. This local disturbance can cause imbalance in the neighboring nerve tissue, allowing the action potential to propagate along the nerve fiber. As a result of the short lasting disturbance at any given point on the nerve fiber, ionic currents are flowing into and out of the membrane of the nerve cells. It is these membrane action currents, which allow the pickup of nerve activity with electrodes adjacent to the nerve, so-called extracellular electrodes.

If an electrode is placed on a cut nerve ending where the intracellular fluid makes good contact with restricted extracellular fluid, and a second electrode is placed further along the uninjured nerve, the shape of the extracellularly recorded action potential is identical to that of the membrane action potential at the second electrode [R. B. Stein and K. G. Pearson. amplitude and form of action potentials recorded from unmyelinated nerve fibres. J. Theoretical biology 32:539-558, 1971]. FIG. 2, shows the setup for a monopolar recording with a single electrode placed around the nerve. The reference electrode is arranged far away from the recording electrode. Whenever the action potentials propagate underneath the electrode, the associated action currents causes voltage differences that can be picked up by the extracellular electrode. The voltage waveform approaches a scaled version of the action potential, with a scaling factor that depends on the transverse and longitudinal conductivity of the medium surrounding the nerve fibers.

The monopolar configuration has the disadvantage that other biological interference, as for instance caused by adjacent muscle activity, will be indistinguishably picked up between recording and reference electrode. This situation can be greatly improved by recording nerve activity between two adjacent electrodes with an instrumentation amplifier which can greatly reduce any common mode interference as shown in FIG. 3. If the electrodes are aligned parallel to the gradient of the electric interference field, a tiny fraction of the greatly extended biological interference field can be sampled as differential voltage, which is increasing with the inter-electrode distance. But the inter-electrode distance cannot be made arbitrary small, because the wavelength of the action potentials increases with the nerve conduction velocity, and thus requires a larger inter-electrode distance for proper spatial sampling especially for fast conducting nerve fibers.

As previously mentioned, the amplitude of the action potentials recorded with extracellular electrodes is also dependent on the conductivity of the surrounding medium. It was found that the amplitude was proportional to the ratio between extracellular and axioplasmatic (i.e. the ohm'ic resistance inside of the nerve) resistivity [A. L. Hodgkin and W. A. Rushton. The electrical constants of a crustacean nerve fibre. Proc. R. Soc. Med. 134 (873):444-479, 1946].

Researchers have shown that if a nerve is brought into another electrically isolating medium like air (lifted the nerve with the attached hook electrode from the biological medium) or paraffin, the voltages significantly increase [L. Hermann. Untersuchungen ueber die Aktionsstroeme des Nerven: Teil II. Pfluger's Arch. ges. Physiol. 24:246-294, 1881], [K. S. Cole and H. J. Curtis. Membrane Potential of the Squid Giant Axon during current flow. J. Gen. Physiol. 24 (4):551-563, 1941]. This led researchers to the idea of surrounding the recording electrodes by an insulating silastic nerve cuff [R. B. Stein, D. Charles, L. Davis, J. Jhamandas, A. Mannard, and T. R. Nichols. Principles underlying new methods for chronic neural recording. Canadian Journal of Neurological Sciences:235-244, 1975], [J. A. Hoffer and G. E. Loeb. Implantable electrical and mechanical interfaces with nerve and muscle. Ann. Biomed. Eng 8:351-369, 1980].

These cuff electrode arrangements can be produced by molding the electrodes into silastic sheets that are wrapped around the nerve, and closed by a suture. As the silicone cuff is surrounding the recording electrodes, it also reduces the picked up differential interference voltages.

The differential interference can be further reduced by connecting three amplifiers in a double-differential configuration as shown in FIG. 4. This scheme was first introduced by [Pflaum et al. An improved nerve cuff recording configuration for FES feedback control system that utilizes natural sensors. Proc. IFESS, pp. 407-410, 1995] for electroneurographic measurements and was referred to as ‘true-tripolar’ configuration. The principle is based on the fact that interference currents cause instantaneous—and ideally equal—voltage differences that are present in each adjacent electrode pair. Thus, Vt1 and Vt2 are of equal phase (FIG. 4). An additional amplifier can be used to nullify the interference by subtracting Vt1 from Vt2. Even if the amplitudes are not equal, for instance due to the difference in inter-electrode impedances Rt1 and Rt2, the interference can be theoretically nullified by proper adjustment of the gain ratio between G1 and G2.

On the other hand, the superpositions of a great number of action potentials that propagate along the longitudinal neural axis constitute the signal of interest. Their conduction velocity, reaching up to approximately 100 m/s, causes a delay between each bipolar recording of amplifier G1 and G2. If the inter-electrode spacing is sufficient for a given nerve conduction velocity, the phase differences will be large enough to prevent the double differential amplifier from nullifying the nerve signals as well. Under the right conditions (i.e. conduction velocity and inter-electrode spacing), the action potential's peak reaches the center electrode, while the end electrodes are located at the very beginning or the very end of the action potential wave. One amplifier detects the positive rising phase, while the other detects the falling phase. Thus, the double differential amplifier configuration allows the amplification of the desired out-of phase nerve activity, while greatly reducing the instantaneous bioelectric interference.

However, the interference reduction performance might be subject to change if the ratio between gains G1 and G2 is fixed. Changes in the impedance balance between Rt1 and Rt2, as well as non-linear field effects that depend on the location of the interference source [Triantis I. F. & Demosthenous A. The effect of interference proximity on cuff imbalance. February 2006, IEEE Trans. BME, 53(2),p.354-7] might require a re-adjustment of the gains G1 and G2 to maintain the desired interference rejection. The need for an adaptive system that automatically tunes gains G1 and G2 was addressed in [Demosthenous A. et al. Design of an adaptive interference reduction system for nerve cuff electrode recording. April 2004. IEEE Trans. Circuits & Systems 51(4), p. 629-639].

The above described research overview points out the basic principle behind recording nervous activity and points out methods for the rejection of undesired bioelectric artifacts, as e.g. those being attributed to muscular activity. The limitations in the previously described methods are the lacking ability to emphasize the nerve signal propagation direction of interest while at the same time being able to reduce the amount of encountered interference. The above described methods are based on arithmetic operations on signals from pairs of electrodes that are carried out by hardware, before sampling and converting the signal into the digital domain. The drawback is that only the signal from one channel can be preserved and thus no information on nerve propagation direction and/or signal velocity can be obtained.

### DESCRIPTION OF THE INVENTION

It is thus an object of the present invention to provide an implantable system for sensing and recording of nerve signals in which the signals can be separated by their propagation direction along the longitudinal nerve axis, by which it becomes possible to discriminate between sensory or motor related activity. Usually only either of which is essential for a particular application, while the other constitutes an undesired neural interference signal.

This is according to the invention achieved by providing a system for recording neural activity comprising at least three electrodes that are adapted to be arranged along the longitudinal axis of a peripheral nerve and further includes means for amplifying and processing the sensed nerve activity where the system is further equipped with means for emphasizing neural signal activity in the neural propagation direction of interest.

More detailed the system comprises at least three equally spaced electrodes that are adapted to be arranged along the longitudinal axis of a concerned nerve. The electrodes can be formed as extracellular electrodes that are either adapted to be placed circumferentially around the nerve, or which are adapted to be placed in-between or even within the individual nerve fascicles.

The electrode arrangement is in an embodiment configured to be used to provide at least two separate bipolar recording channels, where a first signal is recorded from a first bipolar channel formed between the first and second electrodes. The second bipolar channel may be derived between the second electrode and a third electrode.

In a further embodiment, a time delay which is inversely proportional to the conduction velocity of the neural signal is added to the signal recorded from the bipolar channel which is first being passed by the said neural signal. Practically, the delay may be determined as the amount of time it takes a specific neural signal to propagate between the geometric centres of the first and second bipolar channels, respectively.

In an embodiment, the delayed signal from the first bipolar channel is added to the second bipolar channel to amplify sensory information in the principal direction from the first (delayed) channel to the second (un-delayed) channel.

Hereby the sum of both channels will reach a maximum for all action potentials of the same direction and velocity. The output will be reduced for action potentials of different propagation velocity, and especially for those action potentials travelling in opposite direction, from the second to the first bipolar channel. It is thus possible to emphasize nerve activity in a neural propagation direction of interest.

If so desired the first channel may in an embodiment be left un-delayed and the second (opposite) channel may be delayed before addition to amplify information in the opposite direction.

In a further embodiment, the delayed signal from the first bipolar channel may instead be subtracted from the second bipolar channel to attenuate neural information in the principal direction from the first (delayed) channel to the second (un-delayed) channel. Hereby it is possible to attenuate nerve activity in a neural propagation direction of interest.

If so desired the first channel may in an embodiment be left un-delayed and the second (opposite) channel may be delayed before subtraction to attenuate information in the opposite direction.

In a further embodiment, the delay may be implemented by sampling at the predetermined rate, where the sample switch of the first channel, which is first being passed by the neural signal of interest, will be activated by a delay corresponding to the propagation time later than the sample switch of the second channel followed by either addition or subtraction of the resulting signals. The delays may be fine-tuned irrespective of the sampling rate by programmatically controlling the analog-to-digital-converters (ADCs) used for the implementation.

In another embodiment, the delay may also be implemented by sampling both bipolar channels with a common sampling-clock at a rate much higher than the Nyquist rate of the signal where the delay is generated in the digital domain, by awaiting a number of sampling clock cycles before processing.

Further, in yet another embodiment, the instantaneous energy content of the neural activity may be evaluated as the moving variance of the recorded, added or subtracted signals according to Equ. 1 to prepare for a following step of detection of activity:

$${Mov\_var}_{W} ( t ) = \frac{1}{{N}_{W} - 1} \cdot {\sum }_{i = t - {N}_{W}}^{t} {( {x}_{i} - \overline{x} )}^{2} \qquad Equ . ( 1 )$$

where xᵢ denotes the summed or subtracted signal and N_{W} is the number of samples in the sliding window.

Alternatively, in a further embodiment a signal very similar to the moving variance signal may be obtained by directly incorporating the delay and sum operations into a cross correlation operation, where the cross correlation between the two un-delayed bipolar channels is calculated with a time lag corresponding to the desired delay between the channels according to Equ. 2:

R(x, y)=Σₗx*(t)y(t+τ) Equ. (2)

where x(t) and y(t) denotes the two undelayed bipolar channels and τ denotes the desired lag (delay).

The system may give input to any system that aims to react on nerve signals. Especially appreciated will the system be used for giving input to a system for correcting gait related deceases as e.g. drop-foot or to a system for the control of prostheses substituting functional body parts such as artificial legs or arms.

The system can in a further embodiment be used for giving input to a system for the treatment of incontinence.

For all embodiments, the described electrode arrangement or the entire system may be adapted to be implanted in the human or animal body. However it might also be adapted to be arranged outside the human or animal body.

### DESCRIPTION OF THE DRAWING

FIG. 1 shows an illustration of a leg region of a patient with dedicated electrodes implanted for recording nerve signals from the sural nerve, a purely sensory nerve. It also illustrates the placement of a cuff electrode located on the peroneal nerve, for combined stimulation and sensing,

FIG. 2, shows a simplified illustration of a nerve for explanation of the problem of biological interference in monopolar recordings,

FIG. 3, shows a simplified illustration of a nerve for explanation of the problem of both common-mode and differential-mode interference voltages at the input of an instrumentation amplifier,

FIG. 4 shows a simplified illustration of a single-channel cuff electrode placed around the nerve, being subjected to an electric interference field, which can be greatly reduced by the true-tripolar configuration as shown,

FIG. 5 shows the front-end for the implementation of the present invention based on programmable timing of sampling,

FIG. 6 shows the front-end for the implementation of the present invention based on programmable delays in the digital domain,

FIG. 7 shows an example of signals recorded from one bipolar channel of a cuff electrode during sensory activity in a porcine median nerve. The moving variance of the signal reflects the energy content of the signal,

FIG. 8 shows the same signal as in FIG. 7, but one of the two bipolar channels was delayed as described and summed together to emphasize the sensory activity. The energy content of the sensory activity is amplified this way and

FIG. 9 shows the same signal as in FIG. 7, but one of the two bipolar channels was delayed as described and subtracted to reduce the sensory activity. The energy content of the sensory activity is attenuated this way.

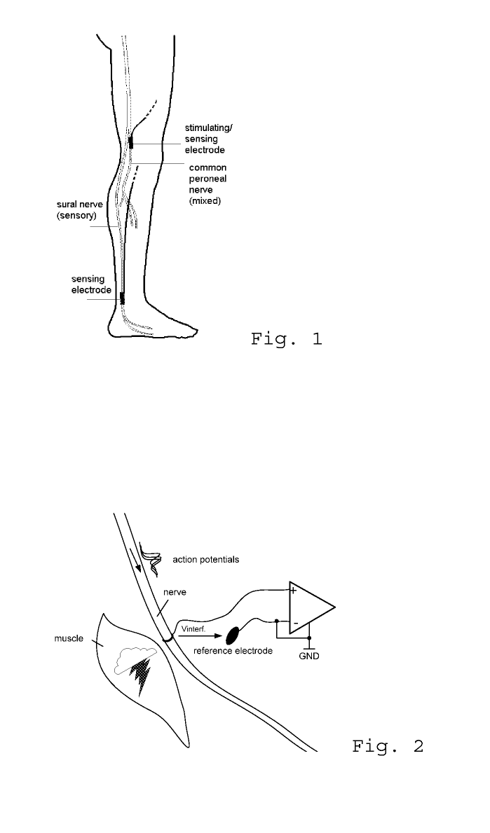

A first number of embodiments, not forming part of the invention but being useful for the understanding of the invention, has already been explained with reference to FIGS. 1 to 4 in the preamble of this application.

The limitation in the previously described methods is the lacking ability to emphasize the nerve signal propagation direction of interest while at the same time being able to reduce the amount of encountered interference. The methods are based on arithmetic operations on signals from pairs of electrodes that are carried out by hardware, before sampling and converting the signal into the digital domain.

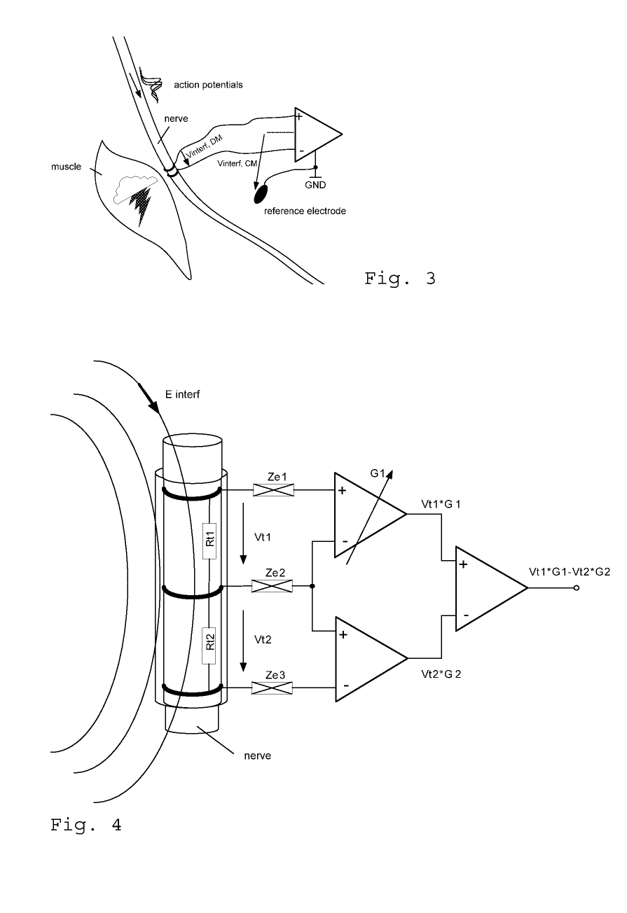

Using the embodiment in FIG. 4 as a starting point, the present invention concerns a system for recording neural activity comprising at least three electrodes that are arranged along the longitudinal axis of a peripheral nerve and means for amplifying and processing the sensed nerve activity and further the system is equipped with means for emphasizing activity in the neural propagation direction of interest.

More detailed the present invention provides an implantable system for sensing and recording of nerve signals in which the signals can be separated by their propagation direction along the longitudinal nerve axis, by which it becomes possible to discriminate between sensory or motor related activity. Usually only either of which is essential for a particular application, while the other constitutes an undesired neural interference signal.

In a preferred embodiment the system comprises at least three equally spaced electrodes that are arranged along the longitudinal axis of the concerned nerve. The electrodes are typically extracellular electrodes that are arranged circumferentially around the nerve, or which are arranged in-between or even within the individual nerve fascicles.

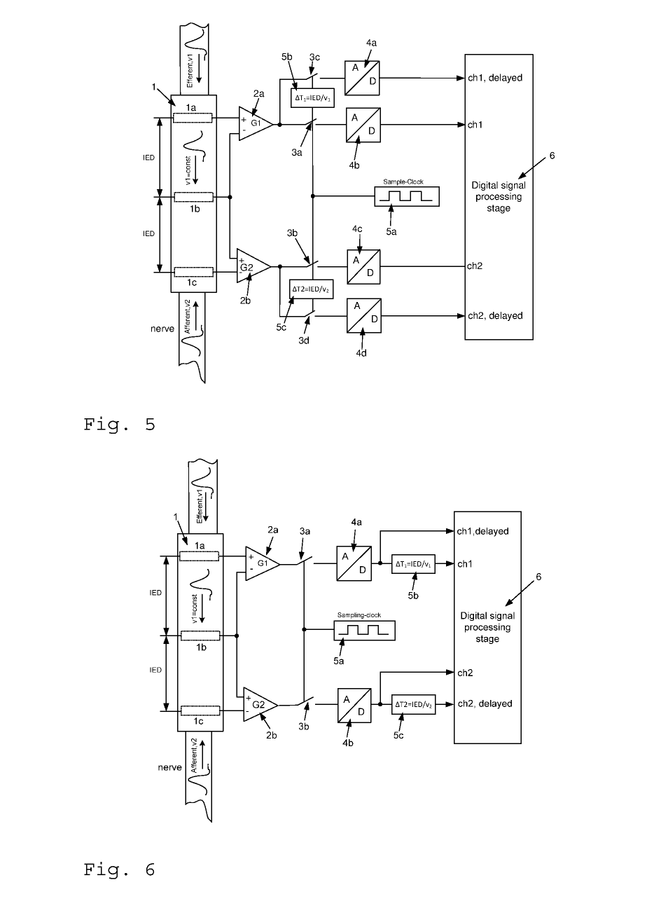

In the embodiment shown in FIG. 5, a cuff electrode arrangement is placed on a peripheral nerve and the shown electrode triplet consists of the electrodes 1a, 1b and 1c. Here, electrode 1a is closer to the spinal cord than electrode 1c, which means that action potentials traveling from electrode 1a to electrode 1c are ‘efferent’ (motor commands), and action potentials traveling the opposite directions are ‘afferent’ (sensory signals).

The electrodes are spaced by the inter-electrode distance IED, with the consequence that the same waveform of the efferent action potential appears at the channel G2, with a delay corresponding to the propagation velocity v1 of the action potential arriving from the spinal cord, ΔT=IED/v1.

The amplifier stages 2a and 2b are both low-noise instrumentation amplifiers that provide sufficient gain and common mode rejection for subsequent sampling 3a, 3d and analogue-to-digital conversion ADCs 4a, 4d. The ADCs are clocked by a common sampling clock 5a with a frequency sufficiently high to avoid aliasing of the recorded nerve signal.

In another embodiment, the channels will be sampled by switches 3a and 3b at the predetermined rate. However, sample switch 3c will be activated at the time ΔT1=IED/v1, 5b later when the efferent activity is to be recorded, and switch 3d is delayed by ΔT2=IED/v2, 5c if afferent activity should be emphasized. In any case, all these cases can be obtained during a single sample clock cycle, and further processed in the digital domain 6. The ADCs can be implemented as two single ADCs with an attached programmable input multiplex stage.

In another embodiment shown in FIG. 6 the said delay is implemented by sampling both bipolar channels with a common sampling-clock 5a at a rate much higher than the Nyquist rate of the signal where the delay is generated in the digital domain 5b, 5c, by awaiting a number of sampling clock cycles before processing.

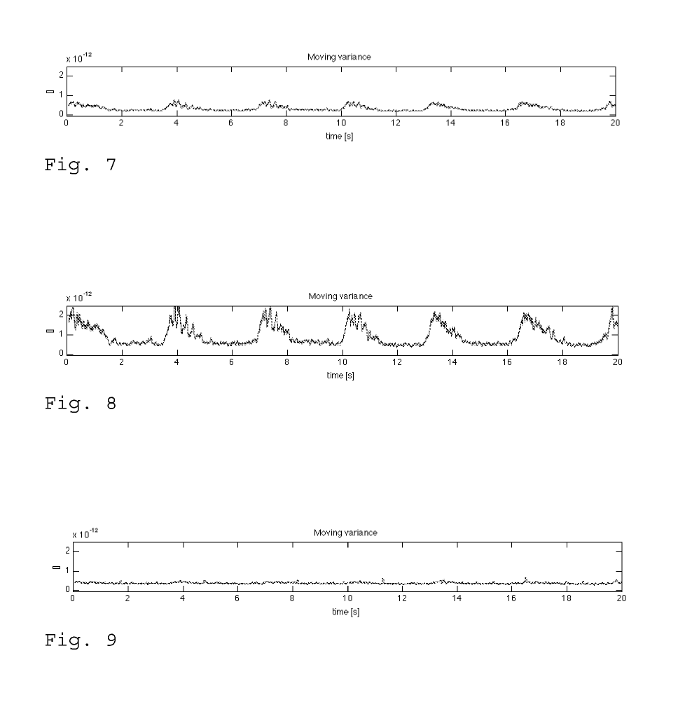

Practical realization of the invention show good results in an animal trial on Gottingen mini pigs. The result is confirmed by the measurements showed in FIGS. 7 to 9.

FIG. 7 shows an example of signals recorded from one bipolar channel of a cuff electrode during sensory activity in a porcine median nerve. The moving variance of the signal reflects the energy content of the signal.

FIG. 8 shows the same signal as in FIG. 7, but one of the two bipolar channels was delayed as described and summed together to emphasize the sensory activity. The energy content of the sensory activity is amplified this way.

FIG. 9 shows the same signal as in FIG. 7, but one of the two bipolar channels was delayed as described and subtracted to reduce the sensory activity. The energy content of the sensory activity is attenuated this way.

1. A system for recording neural activity, comprising:
at least three electrodes adapted to be arranged along a longitudinal axis of a peripheral nerve;
at least one device to amplify and process nerve signals sensed by the at least three electrodes and to emphasize the neural signals in a neural propagation direction of interest;
wherein a time delay which is inversely proportional to a conduction velocity of the neural signals is added to at least one bipolar channel which is first passed by the neural signals.

2. A system according to claim 1, wherein a delayed signal from a first bipolar channel is added to a second bipolar channel to amplify sensory information in a principal direction from the first (delayed) bipolar channel to the second (un-delayed) bipolar channel.

3. A system according to claim 1, wherein a delayed signal from a first bipolar channel is subtracted from a second bipolar channel to attenuate neural information in a principal direction from the first (delayed) bipolar channel to the second (un-delayed) bipolar channel.

4. A system according to claim 2, wherein the first bipolar channel is left un-delayed and the second (opposite) bipolar channel may be delayed before addition to amplify information in an opposite direction.

5. A system according to claim 3, wherein the first bipolar channel is left un-delayed and the second (opposite) bipolar channel may be delayed before subtraction to attenuate information in the an opposite direction.

6. A system according to claim 1, wherein the time delay is implemented by sampling at a predetermined rate, where a sample switch of the first bipolar channel which is first being passed by the neural signal of interest will be activated by a delay corresponding to a propagation time later than a sample switch of the second bipolar channel.

7. A system according to claim 1, wherein the time delay is implemented by sampling at least one bipolar channels with a common sampling-clock at a rate being much higher than a Nyquist rate where the delay is generated in a digital domain, by awaiting a number of sampling clock cycles before processing.

8. A system according to claim 1, wherein an instantaneous energy content of the neural activity is evaluated as the moving variance of recorded, added or subtracted neural signals to prepare for a following step of detection of activity.

9. A system according to claim 1, wherein a signal similar to a moving variance signal may be obtained by directly incorporating the time delay and sum operations into a cross correlation operation, where the cross correlation between two un-delayed bipolar channels is calculated with a time lag corresponding to a desired delay between the un-delayed bipolar channels.

10. A system according to claim 1, wherein the system is configured to provide input to a system for correcting walking disabilities such as drop-foot.

11. A system according to claim 1, wherein the system is configured to provide input to a system for control of prostheses substituting functional body parts such as artificial legs or arms.

12. A system according to claim 1, wherein the system is configured to provide input to a system for treatment of incontinence.

13. A system according to claim 1, wherein the system is adapted to be arranged outside a human or animal body.

14. A system according to claim 1, wherein the system is adapted to be implanted into a human or animal body.

15. A system for recording neural activity, comprising:
at least three electrodes configured to be positioned longitudinally along a peripheral nerve;
at least one device configured to amplify and process nerve signals sensed by the at least three electrodes and for emphasizing the neural signals in a neural propagation direction of interest;
wherein a time delay, which is inversely proportional to a conduction velocity of the neural signals, is added to at least one bipolar channel through which the neural signals pass.

16. A system according to claim 15, wherein a delayed signal from a first bipolar channel is added to a second bipolar channel to amplify sensory information in a principal direction from the first bipolar channel to the second bipolar channel.

17. A system according to claim 15, wherein a delayed signal from a first bipolar channel is subtracted from a second bipolar channel to attenuate neural information in a principal direction from the first bipolar channel to the second bipolar channel.

18. A system according to claim 16, wherein the first bipolar channel is left un-delayed and the second (opposite) bipolar channel may be delayed before addition to amplify information in an opposite direction.

19. A system according to claim 17, wherein the first bipolar channel is left un-delayed and the second bipolar channel may be delayed before subtraction to attenuate information in an opposite direction.

20. A system according to claim 15, wherein the time delay is implemented by sampling at a predetermined rate, wherein a sample switch of the first bipolar channel which is first being passed by a neural signal of interest will be activated by a delay corresponding to a propagation time later than a sample switch of the second bipolar channel.
